(19) United States
(12) Patent Application Publication
(10) Pub. No.: US 2019/0328503 A1
(43) Pub. Date: Oct. 31, 2019

(54) TRANSPLANTED CELL CONTAINMENT AND NUTRITION DEVICE

(71) Applicants: Michael J. Dalton, Evanston, IL (US); Jordan M. Dalton, Libertyville, IL (US); Natan A. Pheil, Chicago, IL (US)

(72) Inventors: Michael J. Dalton, Evanston, IL (US); Jordan M. Dalton, Libertyville, IL (US); Natan A. Pheil, Chicago, IL (US)

(21) Appl. No.: 16/504,597

(22) Filed: Jul. 8, 2019

Related U.S. Application Data
(63) Continuation-in-part of application No. 14/796,927, filed on Jul. 10, 2015, now Pat. No. 10,342,961.
Application No. 14/796,927 is a continuation-in-part of application No. 14/182,418, filed on Feb. 18, 2014, now Pat. No. 10,251,994.
(60) Provisional application No. 61/766,111, filed on Feb. 18, 2013.

(51) Int. Cl. A61F 2/02; A61M 39/02; A61M 39/24; A61L 27/38; A61L 27/54

(52) U.S. Cl. CPC A61F 2/022; A61M 39/0208; A61F 2002/009; A61L 27/3834; A61L 27/54; A61M 39/24

(57) ABSTRACT

The disclosure provides an implanted device that promotes the protection and maintenance of transplanted cells in a host body. The implanted device provides the transplanted cells with a safe, nutritious environment for survival and removes waste products generated by the cells.

### CROSS-REFERENCE TO RELATED APPLICATIONS

This application is a continuation-in-part of U.S. patent application Ser. No. 14/796,927, filed on Jul. 10, 2015, which is a continuation-in-part of U.S. patent application Ser. No. 14/182,418, filed on Feb. 18, 2014, which claims the benefit of U.S. Provisional Patent Application No. 61/766,111, filed on Feb. 18, 2013, and which also claims the benefit of U.S. Provisional Patent Application No. 62/022,795, filed on Jul. 10, 2014. Said applications are incorporated herein by reference in their entireties.

### BACKGROUND

All living cells in the body require three things to survive 1) oxygen, 2) nutrition, and 3) hydration. Ideally, in addition to nutrition, the waste products from the transplanted cells should be removed as needed. In the living body, both human and animal, these functions or requirements are supplied by the interstitial fluid that surrounds all cells. In the case of transplanted cells, these cells additionally need to be protected from the immune system of the host. Therefore, these cells need a protected filtered environment that prevents the components of the immune system from destroying them.

The present invention is directed to an apparatus or device that provides both immuno-protection and nutrition to transplanted cells. The invention is comprised of three (3) separate chambers and mechanisms that when combined provide a system for immuno-protection and for providing fluids required to sustain life.

Diabetes is a group of diseases characterized by high levels of blood glucose resulting from defects in insulin production, insulin action or both. The major types of diabetes include type 1 diabetes, type 2 diabetes, gestational diabetes, and pre-diabetes. Type 1 diabetes, also referred to as insulin-dependent diabetes mellitus (IDDM) or juvenile-onset diabetes, results when the body's immune system destroys insulin producing pancreatic beta cells. Type 1 diabetes accounts for approximately 5-10% of all diagnosed cases. Type 2 diabetes, also referred to as non-insulin dependent diabetes mellitus (NIDDM) or adult-onset diabetes, results from insulin resistance, combined with relative insulin deficiency. Type 2 diabetes represents approximately 90% of all diagnosed cases.

Diabetes mellitus is a chronic debilitating disease affecting over 170 million people worldwide, 5-10% of which, about 8.5 to 17 million, are type 1 diabetic patients. Diabetes is one of the leading causes of blindness, end-stage renal failure, non-traumatic limb amputations, and cardiovascular morbidity and mortality. Quality of life for diabetic patients is evidently decreased, not only in manifestation of complications, but also in managing the disease and fear of life threatening glycemic events.

Patients with type 1 diabetes must have insulin delivered via injection or pump in order to survive. In its early stages, some people with type 2 diabetes can manage the disease through a combination of drugs that increase pancreatic insulin, or act on the liver, muscle or intestine, plus lifestyle changes in diet and exercise. However, despite these efforts, 40% of all type 2 diabetic patients eventually require injections of insulin.

The most promising treatment for both type 1 and type 2 diabetes may be the replacement of damaged pancreatic beta cells with intact functioning beta cells through beta islet transplantation. Cell-based therapies, replacement of the insulin producing pancreatic-cells by transplanting isolated human islet cells intraportally is an approach that has shown remarkable promise in restoring normoglycemia. In turn islet allotransplants have reduced the incidence for frequent and life-threatening complications associated with metabolic instability. However, a shortage of human donor pancreases exists, limiting the number of patients that can take advantage of this therapy. Exacerbating this are intraportal islet transplant protocols that require high islet numbers as a consequence of the significant loss of islets (over half is speculated) immediately post-transplant to hypoxia, inflammation, and immune-mediated loss. There is general agreement that the liver may not be the ideal environment for islets because of exposure to high concentrations of glucagon, diabetogenic immunosuppressive drugs, and toxins from the gastrointestinal tract.

However, there remain major limitations associated with the therapy—i.e. the loss of beta cell viability and function due to the lack of a blood supply and oxygen and nutrients to support cell viability. The present invention is directed to address such limitations.

The present invention is comprised of: an interstitial fluid accumulation chamber/area; a pumping mechanism that will transfer the fluid from the interstitial accumulation chamber/area to the cell containment chamber, and a cell containment chamber/isolation chamber designed to contain the transplanted cells and protect them from the host's immune system. These three mechanisms may be individual components that may be implanted separately in various areas of the body or may be combined in one single structure. Thus, the invention may be constructed such that the mechanisms may be constructed in a single structure or as three separate components wherein each component is connected via a pathway or connector.

The present invention uses the combination of three mechanisms in a novel and unique manner. The present invention comprises a singular, compact device combining three separate mechanisms in a manner that would provide isolation, nutrition, and oxygen to the transplanted cells. While it is important to isolate and protect the transplanted cells, one of the innovative features of this invention is the use of an interstitial fluid accumulation chamber that is placed in the subcutaneous space to collect interstitial fluid and a pumping mechanism to deliver the collected interstitial fluid to the cells contained in the isolation chamber. The combination of the three mechanisms in one system would provide the transplanted cells with a safe, nutritious environment for survival and would also be suitable for removal of the waste products generated by the cells. An innovative feature of this design is to use the host's interstitial fluid, suitably filtered to remove any potentially fatal immune system cells, to sustain the transplanted cells.

The interstitial fluid that occurs in the subcutaneous space is believed to have abundant oxygen and nutrition to allow the transplanted cells to survive and flourish. Such findings have been found in the testing of Lewis rats. A unique aspect of this design is that the cells will be supplied with adequate oxygen and nutrition from the interstitial fluid while in the isolation chamber. The interstitial fluid will be collected and pumped to the transplanted cells. The degree and timing of the pumping of the fluid into the isolation chamber will be dependent on three issues 1) the concentration of oxygen and nutrition in the interstitial fluid; 2) the needs of the cells; and 3) the production of insulin required by the host. The amount and frequency of the pumping and fluid delivery may be fully controllable.

Another innovative aspect of this invention is to include a “port” connected to the isolation chamber such that the transplanted cells can be delivered to the isolation chamber after the device has been allowed to stabilize within the host's body. Additionally, with this port or access point, the quality of the cells and the fluid within the chamber can be easily monitored as well. The “port” or implanted vascular access device is a well-known product used in animal research and in human oncology for long-term vascular access. A port or vascular access device is comprised of a puncture-capable silicone rubber septum placed over or covers an accumulation chamber or reservoir with an outlet. This design allows for a needle of appropriate size to be inserted into the chamber through the septum to deliver the contents of an attached syringe or to remove fluid from the chamber. When the needle is removed, the puncture point of the septum closes to provide a sealed environment within the chamber. The use of a “port” is a unique feature of the present invention. It allows the researcher or physician to monitor, adjust, remove, and/or reintroduce the cells or to simply add drugs to further enhance or adjust the health of the cells contained in the isolation chamber.

The design of the present design is such that the insulin produced by the cells in the isolation chamber can be delivered into the subcutaneous environment or to a remote site if that is deemed the best therapeutic approach. Insulin could be delivered to the peritoneal cavity or the portal vein or any other suitable area or vessel through the use of a special silicone catheter placed at the outlet of the Isolation or cell containment chamber of the present invention.

The overall design of the present invention is to provide the transplanted/isolated cells with adequate amounts of oxygen and nutrient containing fluid to maintain cell integrity. The present invention further removes the cellular waste along with the insulin that is washed from the isolation chamber on an as needed basis.

All components may be fabricated of biocompatible and pliable elastomeric material preferably Medical grade silicone rubber or elastomeric material and medical grade metals such as Titanium or stainless steel.

The present invention is also directed to a method and apparatus for treating varying conditions of mammals using transplanted cells to produce matter which is distributed within the mammals to treat the conditions.

### SUMMARY

In one embodiment, a method of producing and delivering matter within a mammal is disclosed. In one step, an apparatus is inserted within a mammal. The apparatus comprises an accumulation chamber, a pump, and an isolation chamber. In another step, interstitial fluid is flowed within the mammal into the accumulation chamber. In still another step, the interstitial fluid is pumped, with the pump, from the accumulation chamber into the isolation chamber to provide nutrients to transplanted cells disposed within the isolation chamber. In yet another step, the matter is produced with the transplanted cells disposed within the isolation chamber. In another step, the matter is pumped from the isolation chamber to a desired location within the mammal to treat anemia, chronic pain, fabry disease, hearing loss, hemophilia, renal failure, chronic liver disease, or a neurological disease.

The scope of the present disclosure is defined solely by the appended claims and is not affected by the statements within this summary.

### BRIEF DESCRIPTION OF THE DRAWINGS

FIG. 1 is a top view of an embodiment of the present invention.

FIG. 2 is a side view of an embodiment of the present invention.

FIG. 3 is a cross sectional view of an embodiment of the present invention.

FIG. 4 is a perspective view of an embodiment of the present invention.

FIG. 5 is an isometric view of an embodiment of the present invention.

FIG. 6 is an isometric view of an embodiment of the present invention.

FIG. 7 is an isometric view of an embodiment of the present invention.

FIG. 8 is an isometric view of an embodiment of the present invention.

FIG. 9 is an isometric view of an embodiment of the present invention.

FIG. 10 is an isometric view of an embodiment of the present invention.

FIG. 11 is an isometric view of an embodiment of the present invention.

FIG. 12 is a schematic view of the present invention.

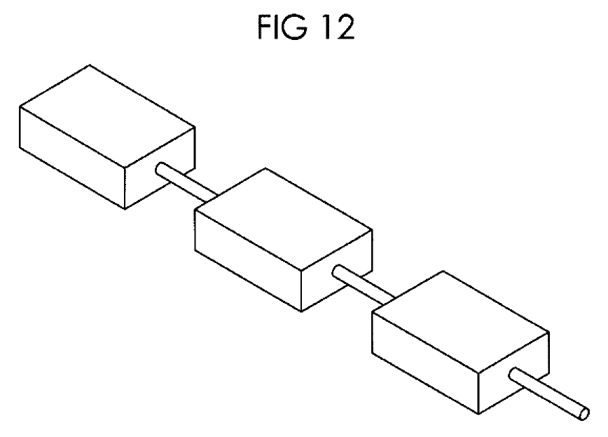

FIG. 13 is an isometric view of an embodiment of the present invention.

FIG. 14 is an isometric view of an embodiment of the present invention.

FIG. 15 is an exploded isometric view of an embodiment of the present invention.

FIG. 16 illustrates one embodiment of a method of producing and delivering matter within a mammal.

### DETAILED DESCRIPTION

This present invention provides an implanted device that promotes the protection and maintenance of transplanted cells in a host body.

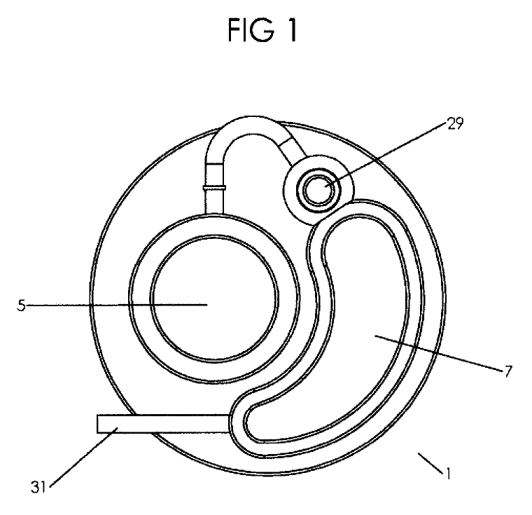

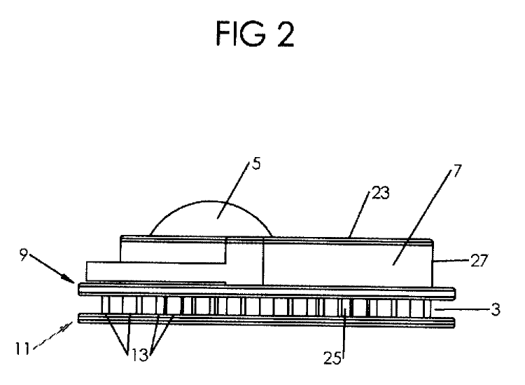

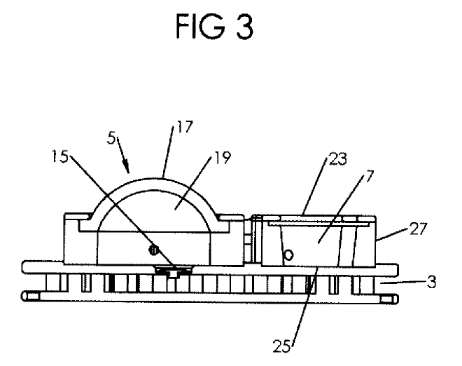

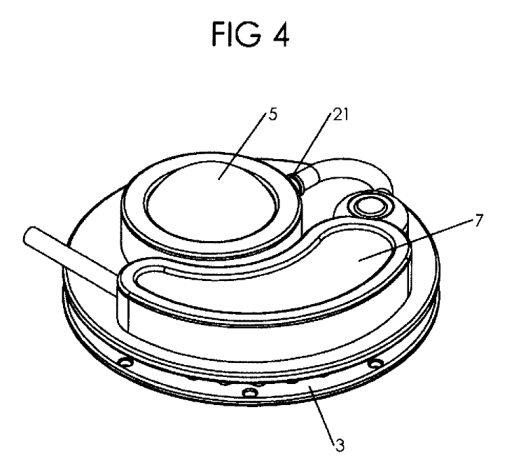

In one embodiment of the present invention, as illustrated in FIGS. 1, 2, 3 and 4, the invention shown generally as 1 is comprised of an interstitial fluid accumulation chamber/area 3, a pumping mechanism 5 that will transfer the fluid from the interstitial accumulation chamber/area to the cell containment chamber/isolation chamber 7, and a cell containment chamber/isolation chamber 7 designed to contain the transplanted cells and protect them from the host's immune system.

### Interstitial Fluid Accumulation Area

Cells in a living body obtain their nutrition and oxygen from the interstitial fluid around them. The nutrition and oxygen in the interstitial fluid is transferred there by the host's blood stream and delivered to the cells in the interstitial fluid. The interstitial fluid contains enough oxygen and nutrition to keep the cells alive and flourishing as though they were in a compatible environment. To supply transplanted cells with the necessary nutrients and oxygen supply, the present invention provides an isolated protected chamber, mechanism or area that allows all of the required fluid to be accumulated. Interstitial fluid needs to be accumulated in a specific area and transferred to the cell containment chamber 7 on an as needed basis. The present invention addresses such need by creating a cavity or space in the subcutaneous tissue via the interstitial fluid accumulation chamber, mechanism or area 3. The accumulated interstitial fluid is then pumped to a cell containment area 7 via a pumping mechanism 5.

The fluid accumulation within the interstitial fluid accumulation chamber, mechanism, area 3 will occur rapidly and continuously over time and be sufficient to supply all of the nutritional needs of the transplanted cells.

In one embodiment of the present invention, the interstitial fluid accumulation chamber 3 is a chamber having a top plate 9 and a bottom plate 11 held apart by a plurality of posts 13 that extend from the top surface of the bottom plate 11 to the bottom surface of the top plate 9. The area between the top plate 9 and the bottom plate 11 is determined by the volume of the fluid that needs to be accumulated. The plurality of posts 13 are placed around the periphery or outer edge of the accumulation chamber, mechanism area 3 to create a tortuous path for tissue to grow into and prevent the tissue ingrowth from totally penetrating the center space of the chamber 3. The distance between the plates is predetermined to prevent tissue from growing over the plates and occluding them.

The top and bottom plates 9 and 11 may be fabricated of pliable silicone rubber. The top and bottom plates 9 and 11 are held apart by a plurality of posts 13 or columns in the interior of the disk. The inlet/outlet catheter 15 is in the center of the disk and is protected from the host's invading tissue by the posts that create a torturous path for tissue invasion and preventing the center from being engulfed. The top and bottom plates 9 and 11 are spaced apart such that the tissue that would engulf a catheter or a small device will not grow across the gap created between the top and bottom plates 9 and 11. While the top and bottom plates 9 and 11 of the disk may become engulfed with tissue, the center of the device will remain open and allow for fluid accumulation.

In another embodiment, the posts 13 or columns between the top 9 and bottom plates 11 may be replaced with a suitable filter material or a metallic screen or mesh or plurality of such materials. The purpose of these elements is simply to prevent tissue from growing into the center space.

In another embodiment, filters or tissue preventing mesh or means may be used in the accumulation chamber/area 3 in order to prevent tissue from growing into the center space of the accumulation chamber/area 3. In one embodiment, filters may replace the outlet/catheter 15 in the isolation chamber 7 to allow fluid to be dispersed into the interstitial space and to keep the isolated cells from escaping the isolation chamber 7.

Also it is important for the fluid that is accumulated to be free from debris and the defense mechanisms of the host. In one embodiment, a filter may be included around the center of the interstitial fluid accumulation chamber 3 to protect the cells from the hosts' immune system's killer cells.

Once adequate interstitial fluid has been accumulated in the accumulated interstitial fluid chamber 3, such interstitial fluid must be flowed to the pumping mechanism 5. To accomplish this, a catheter or outlet 15 is positioned at the bottom center of the bottom plate 11 or in the center of the top plate 9 of the accumulated interstitial fluid chamber 3 providing a pathway to the pumping mechanism 5. The bottom plate 11 of the accumulated interstitial fluid chamber 3 is semi-rigid to contour to the host's body structure. The top plate 9 may have an outside ring or diameter of semi-rigid material while the center is thinner elastic silicone rubber.

In one embodiment, the volume of the accumulated interstitial fluid chamber 3 is approximately 4 cc's with the elastic center volume being approximately 3 cc's. As the pumping mechanism 5 is activated, fluid is forced from the pump area 5 into the cell containment chamber 7. When the pump 5 is released, the restoring force of the pumping mechanism will create a negative pressure within the pumping area and due to this negative pressure the elastic top 17 of the accumulated interstitial fluid chamber 3 will be drawn down. With such negative pressure, fluid is withdrawn from the accumulated interstitial fluid chamber 3 into the pump area 5. Due to the elastic nature of the elastomeric material and the restoring force of the pumping mechanism, negative pressure will be maintained until the pump mechanism is filled with interstitial fluid and the system returns to the resting state.

To prevent interstitial fluid from flowing back towards the accumulated interstitial fluid chamber 3, an inlet check valve or simple backflow mechanism is present in the pathway between the accumulated interstitial fluid chamber and the pump area. The negative pressure in the pump area once the pump is released will cause the inlet check valve to stay open and keep the outlet side check valve closed.

### Pump

In the present invention, the pump 5 may be comprised of a chamber having an elastomeric dome 17 that may be depressed or activated to cause interstitial fluid to be withdrawn from the pumping chamber 19 to the cell containment chamber 7.

In one embodiment, the pump 5 will require manual operation. The user will physically press the dome 17 of the pump to move the fluid from inside of the pumping chamber 19 to the cell isolation chamber 7. In one embodiment, the dome 17 may be constructed of a simple silicone rubber dome fabricated of 60 durometer silicone having a wall thickness of 0.010″ to 0.100″ and has a semi-ridged or ridged bottom. The volume of the pumping chamber 19 may be approximately 3 cc's in fluid volume. The pumping mechanism 5 additionally has at least one inlet and at least one outlet 21. The at least one inlet and outlet have one-way valves or check valves to allow flow out of the accumulated interstitial fluid chamber to the pumping chamber 19 and from the pumping chamber 19 to the cell isolation chamber 7. Interstitial fluid flows from the pumping chamber 19 to the cell containment chamber 7 once the dome 17 is depressed or activated and interstitial fluid flows into the dome 17 from the accumulated interstitial fluid chamber 3 once the volume is discharged from the pumping chamber 19. When the positive external pressure is released and the pumping chamber 19 is allowed to refill and return to the resting state, the retaining force of the silicone dome 17 will create a small negative pressure that will draw interstitial fluid into the pumping chamber 19 from the accumulated interstitial fluid chamber 3.

In one embodiment, the pumping mechanism is comprised of a pumping chamber 19, a simple silicone dome 17, and check valves or duck bill valves at both the inlet and outlet of the pumping chamber. Pressing the top of the dome 17 will force the accumulated interstitial fluid from the pumping chamber 19 into the cell containment chamber 7. When the pressure or dome 17 is released, the elastic properties of the flexible dome 17 will draw fluid into the pumping chamber 19 from the accumulated interstitial fluid chamber 3, that is, after the dome 17 is pressed and the fluid is expelled, the elastic restoring force of the dome 17 will create a negative pressure in the pumping chamber 19 and draw fluid from the accumulated interstitial fluid chamber 3 into the pumping chamber 19 over time. The check valves will allow flow in only one direction, i.e., from the accumulated interstitial fluid chamber 3 to the pumping chamber 19 and from the pumping chamber 19 to the cell containment chamber 7.

In another embodiment, the pumping mechanism 5 may employ an electric motor, i.e., a linear peristaltic, a rotary peristaltic or a simple piston metering programmable pump that would more accurately and precisely deliver the accumulated fluid to nurture the transplanted cells. Additionally, the pump 5 could communicate with a glucose sensor in the fluid accumulation chamber to provide feedback and, thus, better control of the insulin needs of the patient. Additionally, the pumping mechanism 5 may be controlled via a computer, micro-processor, sensor or monitor to be engaged or controlled independently of user manual manipulation.

In the case of the type 1 diabetic, until the communication between the vascular system and the cells is established and the delivery of the insulin is automatic, the distinct advantage of the pumping mechanism 5 is that as the insulin is needed, as determined by a glucose sensor, the pump 5 can be actuated to supply the required insulin by the host. Or the pump can be pressed routinely before meals to provide a needed bolus of insulin. After the communication is established, the pump 5 would be activated on some routine basis simply to supply the transplanted cells with oxygen and nutrition and remove cellular waste.

### Transplanted Cell Isolation Chamber/Cell Chamber

The function of the cell containment chamber 7 is to protect and allow the cells contained within it to flourish and be protected from the hostile environment of the host. Much has been written and directed to providing an environment inside a host body where the cells are protected from the body's immune system. The cell containment chamber 7 may be comprised of a biocompatible enclosure or chamber. The cell containment chamber 7 may be constructed specifically of silicone rubber with a top portion 23, bottom portion 25 and periphery walls 27. A portion of the bottom of the chamber 25 may be comprised of a semipermeable filter membrane. This membrane is porous to the fluids produced by the cells yet a barrier to the host's immune system—particularly the host body's NK cells, “T” cells and B cells. Typically the immune system's cells are in the range of 4-12 microns. In one embodiment of the present invention, the bottom of the chamber 25 is constructed of a filter material with pore size 1 micron although other size filters and other placements of the filter are possible. The surface of the bottom portion of the chamber 25 may be enhanced with a hydrogel compound on which the transplanted cells will thrive. In one embodiment, a cellular matrix or scaffold material may be present in the interior of the cell containment chamber 7. The use of such matrix and scaffold material is well known in other devices. The size of the chamber may be typically approximately between 1.0 and 5.0 cc's in volume. This volume is selected for the ability to contain approximately 100,000 to as much as 400,000 islets. In other embodiments, the volume may vary. An amount determined to product enough insulin to maintain glycemic status quo.

In one embodiment, the cell containment chamber 7 may be cylindrical in shape and may be possibly be 1″ in diameter. The top portion 23 may be elastomeric in material to expand to contain approximately 3 cc's of fluid pumped into the cell containment chamber 7 by the pumping mechanism 5. The bottom portion 25 of the cell containment chamber 7 will be comprised of a composite material of an appropriate filter material and a Dacron felt or velour material to promote vascularization close to the cells within the chamber 7. The interstitial fluid that is pumped into the cell containment chamber 7 will diffuse out of the bottom portion of the cell containment chamber 7 through a filter material. When the pumping mechanism 5 is activated, the flexible elastomeric top 23 of the cell containment chamber 7 is expanded due to the increased fluid entering the cell containment chamber 7 and the fluid will be “pushed” out through the porous bottom portion 25 of the cell containment chamber 7 slowly and into the host's interstitial fluid and blood stream. The size and porosity of the bottom portion 25 and filter material will be adjusted to restrict the outflow such that the inflow of interstitial fluid will mix with the cell producing enzymes and flow out as a combined fluid. It is important to not only provide the living cells with nutrients, but also to remove the waste products of the living cells. The flow of fluid through the cell containment chamber 7 will accomplish this.

In one embodiment, the volume of the chamber 7 will be approximately 5 cc's to accommodate about 500,000 Islet of Langerhans cells. In other embodiments, the volume may vary. The top of the cylinder 23 will be covered with flexible, elastic silicone that will expand when the interstitial fluid is pumped into the chamber 7. The memory of the elastic silicone will provide a restoring force on the fluid to push it through the bottom filter, thereby flushing the cells with oxygenated nutritious fluid and carrying the waste products away. The flow and dwell time for the interstitial fluid will be determined by both the pore size and the overall size of the filter window.

In order to monitor the cell containment chamber 7, a small compressed silicone septum window/portal 29 may be placed adjacent to the cell containment chamber 7 to sample fluid and to withdraw and replenish the cells in the cell house if necessary. Alternatively, the septum window/portal 29 may be incorporated in the top surface of the cell containment chamber 7. Additionally angiogenesis drugs can be injected/infused through the portal 29 to promote vascularization outside of the cell house base. Vascularization is important for communications between the cells and the blood stream. Ideally this communication will be both ways, from the cells out and from the vasculature into the cell chamber to elicit the production of the enzymes designated by the transplanted cells.

As illustrated in FIGS. 1, 2, 3, and 4, the interstitial fluid accumulation chamber 3, the pumping mechanism 5 and the cell containment chamber 7 may be constructed in a single housing wherein each component may be adjacent to one another in a linear fashion. In another embodiment, each component may be connected to one another in a singular housing wherein the components form a triangular overall structure. In another embodiment, the components may be connected to one another in a circular fashion such that each component is concentric to one another.

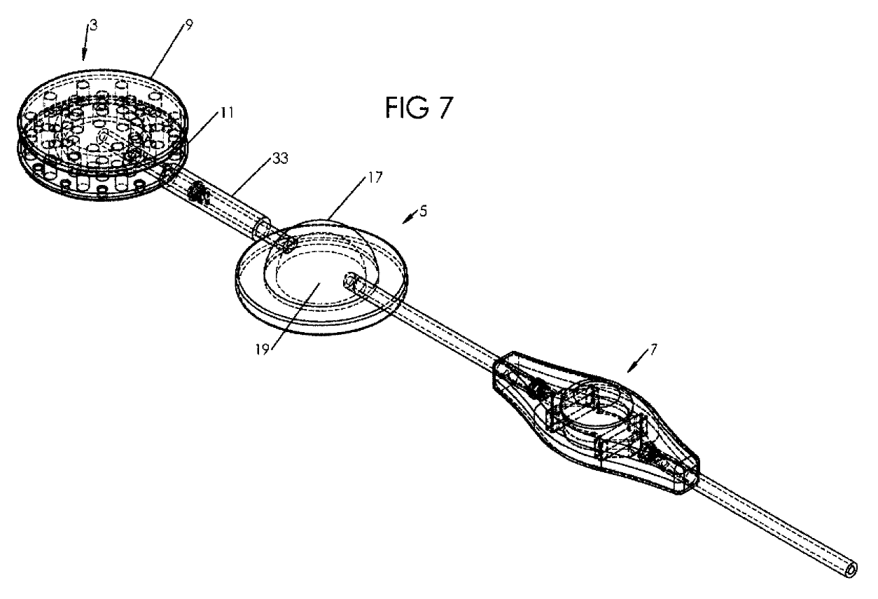

As illustrated in FIGS. 7, 8, 9 and 10, each component may be connected via a pathway in a linear fashion. In one such embodiment, each component may be placed in a different area within the host body.

The overall size of the present invention may be controlled by two elements: 1) the isolation or cell containment chamber size, and 2) the pump capacity. It is estimated that an islet of Langerhans cell count of 500,000 would be required for normal glycemic control in an adult. This amounts to a volume in the isolation chamber of approximately 5 cc's. And 2) the pumping mechanism is designed at an equivalent 5 cc's.

In one embodiment, the device may be round in shape. In such embodiment, the specifications are that the diameter will be less than 2.5″/64 mm in diameter and 0.625″/16 mm in height or thickness. At this diameter and thickness, placement anywhere in the human body is a possibility. For research applications, where a smaller isolation chamber is required that is, less than 5 cc's, a smaller size and different configuration is highly possible.

As illustrated in FIGS. 1, 2, 3, 4, 5 and 6, in one embodiment of the present invention, the interstitial fluid accumulation chamber 3 may be in an area beneath the pumping chamber 5 and is comprised of a top plate 9 and a bottom plate 11 held apart by a plurality of posts 13 that extend from the top surface of the bottom plate 11 to the bottom surface of the top plate 9. In this embodiment, the top plate 9 has an opening 15 leading to the bottom portion of the pumping chamber 19 which has a corresponding opening. Once adequate interstitial fluid has been accumulated in the accumulated interstitial fluid chamber 3, fluid will flow from the interstitial fluid accumulation chamber to the pumping chamber. In order to prevent fluid flowing back to the interstitial fluid accumulation chamber 3, an umbrella style check valve or back flow valve is present in the opening between the top plate 9 of the interstitial fluid accumulation chamber 3 and the bottom portion of the pumping chamber 19.

In one embodiment, when the pumping mechanism 5 is activated or the dome 17 of the pump is depressed the fluid present in the pump 17 will be forced through a port 29 that is connected to the cell containment chamber 7 and then to the cell containment chamber 7. The port 29 may be a standard access port with a reservoir and silicone septum that allows a user to access the reservoir. The cell containment chamber 7 may be a chamber that is connected to an outlet catheter or tube 31. As the pump 5 is depressed, pressure forces the valve between the pumping chamber 19 and port 29 to open to allow fluid to flow from the pumping chamber 19 to the cell containment chamber 7 via the port 29. When the pump 5 is released, negative pressure in the system simultaneously opens the valve or umbrella valve at the bottom portion of the pumping chamber 19 and closes the valve between the pumping chamber 19 and cell containment chamber 7.

In this embodiment, at rest, the interstitial fluid accumulation chamber 3 fills with accumulated fluid and fluid flows from the interstitial fluid accumulation chamber 3 to the pumping chamber 19. In addition, the cell containment chamber 7 is partially filled with nutrient fluid and Islet cells and insulin continues to flow out of the cell containment chamber 7 via a selective filter.

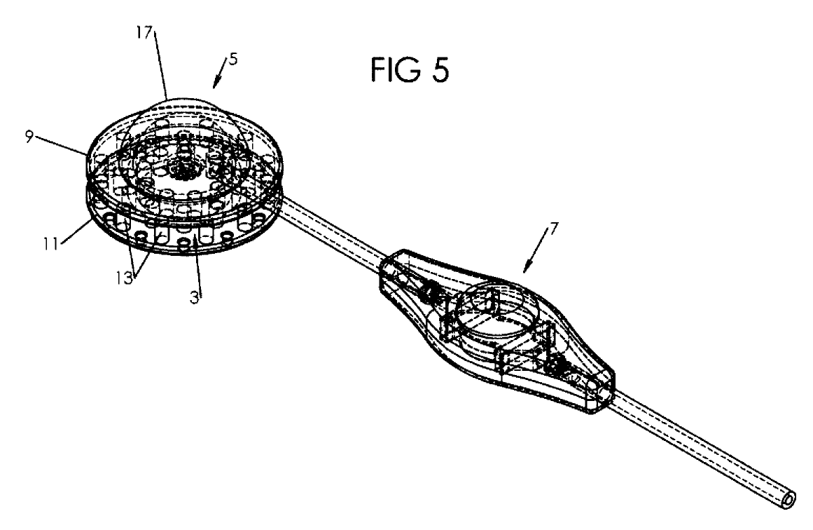

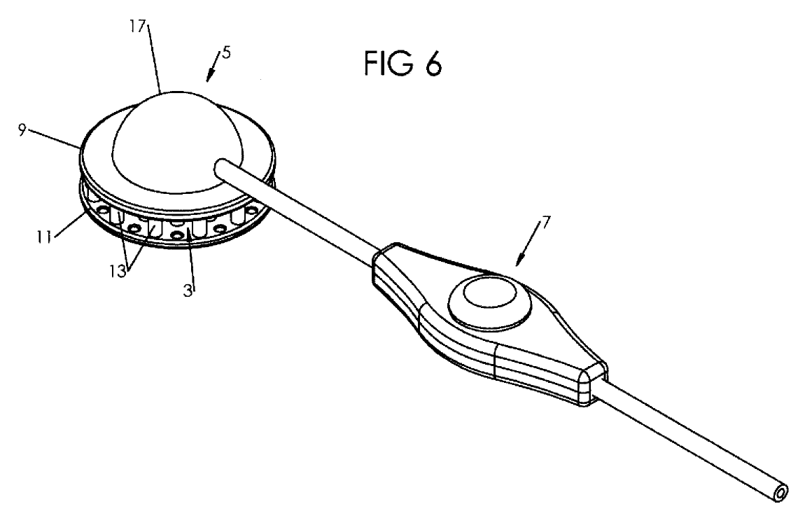

In the embodiment illustrated in FIGS. 5 and 6, the cell containment chamber 7 may not be in the same housing as the pump 5 and accumulation chamber 3. In such embodiment, when the system is at rest, the accumulation chamber 3 fills with nutrient rich interstitial fluid; the pump chamber 19 slowly fills with interstitial fluid as pressure in the accumulation chamber 3 increases. The cell chamber 7 is filled with used nutrient fluid. As the pump dome 17 is depressed, pressure forces open the valve between the pump 5 and accumulation chamber 3 to allow interstitial fluid to flow through to the cell containment chamber 7. New interstitial fluid flows into and fills the cell containment chamber 7 and old interstitial fluid and insulin flows out of the cell containment chamber 7 via the outlet catheter. When the pump 5 is released, negative pressure of the pump 5 release simultaneously opens valve to pump chamber 19 to refill with interstitial fluid and closes valve to the cell containment chamber 7 and insulin flow out of the cell containment chamber 7 ceases.

As illustrated in FIGS. 7, 8, 9, and 10, in another embodiment of the present invention, the interstitial fluid accumulation chamber 3 may be in an area detached from the pumping mechanism 5 and the isolation chamber 7, but connected to the pumping mechanism 5 through connection lines 33 or conduits for the fluid to be pumped through. In this embodiment the device can be two or three separate components as may be the case for the application. As such, the pumping mechanism 5 may be located in an area convenient for the patient's use while the accumulation chamber 3 and the isolation chamber 7 may be located in more suitable locations such as an area that is protected and where abundant interstitial fluid may accumulate.

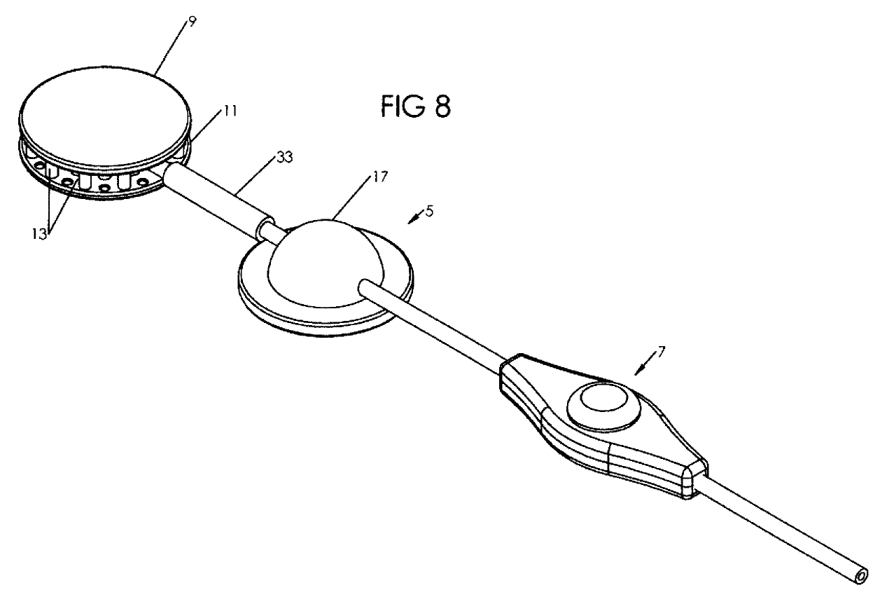

In the embodiment illustrated in FIGS. 7 and 8, the components of the present invention are placed in a linear fashion wherein the accumulation chamber/area 3 is at one end and the cell containment chamber 7 is at the opposite end. In this embodiment, when the system is at rest, the accumulation chamber 3 fills with nutrient rich interstitial fluid; the pump chamber 19 slowly fills with interstitial fluid as pressure in the accumulation chamber 3 increases. The cell chamber 7 is filled with used nutrient fluid. As the pump dome 17 is depressed, pressure forces open the valve between the pump 5 and cell chamber 7 while closing valve between pump 5 and accumulation chamber to allow interstitial fluid to evacuate to cell chamber 7.

New interstitial fluid flows into and fills the cell containment chamber 7 and old interstitial fluid and insulin flows out of the cell containment chamber 7. When the pump 5 is released, negative pressure of the pump 5 release simultaneously opens valve to pump chamber 19 to refill with interstitial fluid and closes valve to the cell containment chamber 7 and insulin flow out of the cell containment chamber 7 ceases.

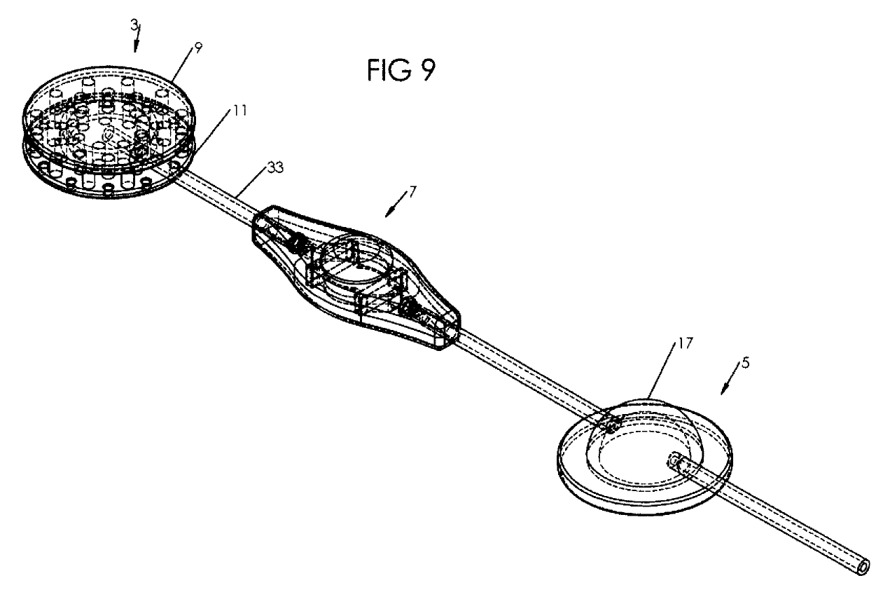

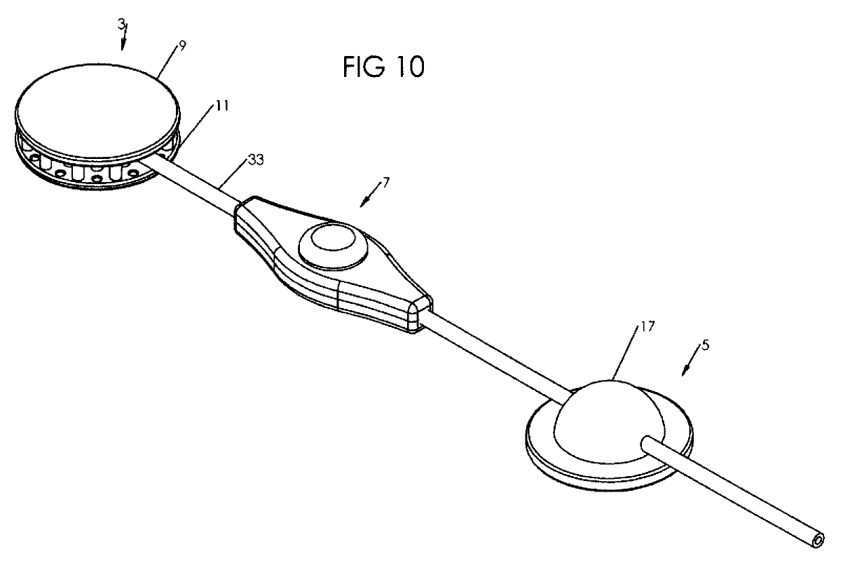

In the embodiment illustrated in FIGS. 9 and 10, the components of the present invention are placed in a linear fashion wherein the accumulation chamber/area 3 is at one end and the pump 5 is at the opposite end. In this embodiment, when the system is at rest, the accumulation chamber 3 fills with nutrient rich interstitial fluid; the cell chamber 7 slowly fills with interstitial fluid as pressure in the accumulation chamber 3 increases. The pump 5 is filled with used nutrient fluid. As the pump dome 17 is depressed, pressure forces used nutrient fluid and insulin in pump 5 to evacuate into the body. When the pump 5 is released, negative pressure of the pump 5 release simultaneously opens all valves allowing interstitial fluid from accumulation chamber 3 to flow into cell containment chamber 7 and interstitial fluid and insulin from cell containment chamber to flow into the pump 5.

In one embodiment, an outlet catheter 31 or tube leads out of the cell containment/isolation chamber 7 to be able to allow a user to have insulin delivered to any desired location in the body. The design of the present invention is innovative in that allows insulin produced by the cells in the cell containment chamber 7 to be delivered to a remote site if that is deemed the best therapeutic approach. Delivery of the insulin could be to the peritoneal cavity or the portal vein through the use of a special silicone catheter placed at the outlet side of the cell containment chamber is possible in this embodiment.

Another innovative aspect of the present invention is the use of a “port” 29 connected to the cell containment chamber 7 such that the transplanted cells can be delivered after the device has been allowed to stabilize within the host's body. With this port 29 or access point, the quality of the cells and the fluid within the chamber 7 can be monitored easily. It also allows researchers or physicians to monitor, adjust, remove, and/or reintroduce the cells or to simply add drugs to further enhance or adjust the health of the cells contained in the isolation chamber 7.

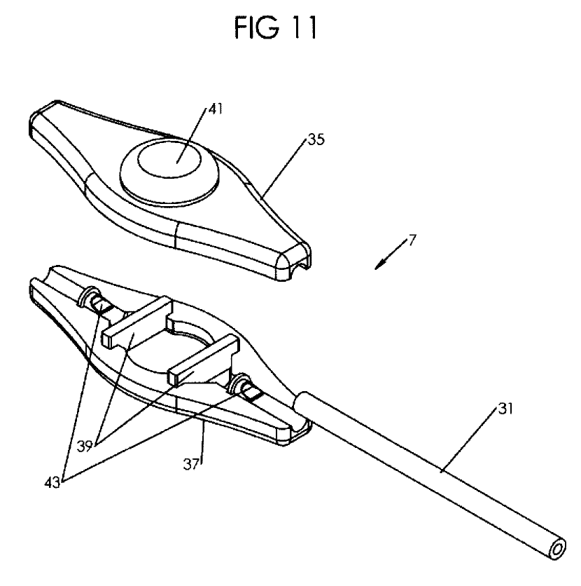

In one embodiment of the invention as illustrated in FIG. 11, the cell containment chamber 7 may have a top body 35, a bottom body 37 and an outlet catheter 31. The top body 35 has a septum 41 which serves as an access point through which the quality of the cells and the fluid within the chamber 7 can be monitored easily. The bottom body 37 has two selective filters 39 and two check valves 43, one at the entrance of the chamber 7 and one at the exit of the chamber 7.

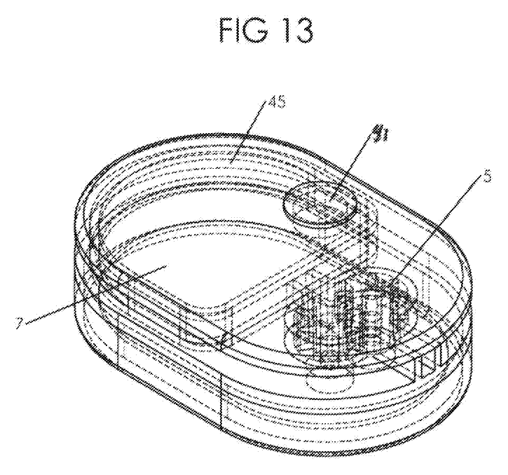

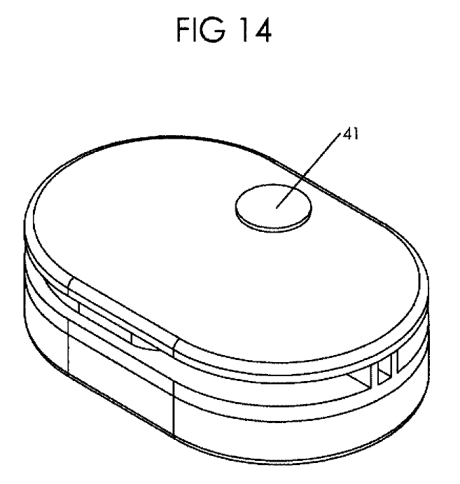

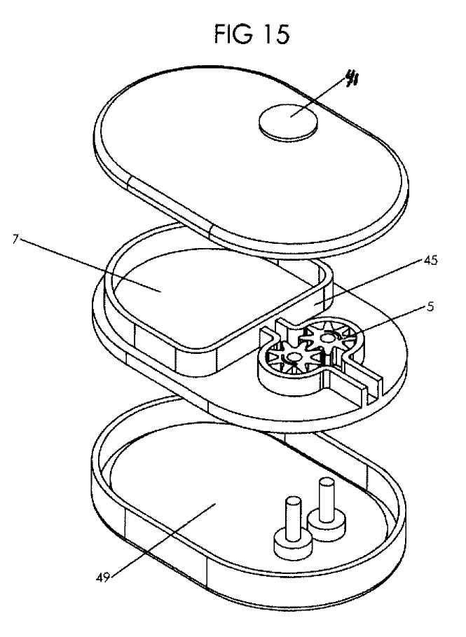

In a preferred embodiment as illustrated in FIGS. 13, 14 and 15, the accumulation chamber 3 and cell containment chamber/isolation chamber 7 occupy the same portion/area of the device. A selective filter 45 surrounds the entire cell containment chamber/isolation chamber 7. A pump 5 moves interstitial fluid out of the cell containment chamber/isolation chamber 7 to the body at a rate sufficient to supply the patient's body with insulin. The selective filter 45 is open to the patient's body which allows interstitial fluid to accumulate in the cell containment chamber/isolation chamber 7. As illustrated in FIGS. 13, 14, 15, the pump 5 may be a set of gears. The pump may be any conventional pumping means. In one embodiment, a circuit board 49 may house the electrical components necessary to control the rate of flow, provide power to the motors and/or recharge a power source. The present embodiment provides a septum 41 which serves as an access point through which the quality of the cells and the interstitial fluid within the cell containment chamber/isolation chamber 7 can be monitored easily.

Although only human applications have been discussed, it should be clear that the present invention may be easily used in any mammal including but not limited to veterinary applications or animal research and can be applied to both therapeutic use and research applications in any living body, human and animal.

In one embodiment, the apparatus of the invention may be used to treat anemia with kidney cells secreting erythropoietin. Anemia is defined as a decrease in the amount of red blood cells, hemoglobin in the blood, or a lowered ability of the blood to carry oxygen. Anemia can be caused by bleeding, insufficient red blood cell production, and red blood cell destruction. Typical causes of blood loss include trauma and gastrointestinal bleeding. Anemia is the most common blood disorder affecting about ⅓ of the global population. Iron-deficiency anemia affects nearly 1 billion people. The apparatus of the invention has been shown to protect foreign cells and provide a nutrient supply while removing waste products to maximize cell viability. Erythropoietin (EPO) is the hormone produced in the kidney that promotes formation of red blood cells by the bone marrow. EPO stimulates the bone marrow to produce more red blood cells. The resulting rise in red cells increases the oxygen-carrying capacity of the blood. EPO's major functions are to promote the development of red blood cells and to initiate the synthesis of hemoglobin, the molecule within red blood cells that transports oxygen. Chemically, EPO is a protein with an attached sugar (glycoprotein) and regulates the blood cells in bone marrow by stimulating differentiation and proliferation. The apparatus of the invention can deliver isolated renal peritubular cells, or engineered cells capable of production and secretion of EPO, in a manner that maintains cell viability and function. The apparatus of the invention provides a cell implantation therapy that protects transplanted EPO-producing cells from the host immune system. Implanted cells result in fewer treatments than the standard of care (multiple injections) which improves patient compliance as well as maintains normal hormone levels, and increases the success rate of EPO treatment in treating anemia.

In another embodiment, the apparatus of the invention may be used to treat chronic pain with implantation of chromaffin cells. Chronic pain is defined as any pain that lasts longer than 3-6 months. Chronic pain can be divided into 2 categories: nociceptive and neuropathic. Nociceptive is caused by the activation of nociceptors and this type of pain can be broken down into multiple levels of pain which include deep somatic pain and visceral pain. Deep somatic pain is characterized as dull aching, poorly-localized pain. Visceral pain can be well-localized but is often extremely difficult to locate.

25 million Americans suffer from chronic pain and there is a serious lack of non-opioid treatments to address unrelieved pain. The apparatus of the invention has been shown to protect foreign cells and provide a nutrient supply while removing waste products to maximize cell viability. It has been discovered that cell therapy using chromaffin cell (CC) transplant at sites of injury releases amines and peptides capable of alleviating chronic pain. The apparatus of the invention can deliver isolated CCs, or engineered cells capable of production and secretion of opioid peptides and catecholamines, in a manner that maintains cell viability and function. The apparatus of the invention can be used as a cell implantation therapy that protects transplanted CC or CC-like cells from the host immune system. Implanted cells result in fewer treatments than the standard of care (repeated dosing of opioid) which significantly de-risks the treatment of chronic pain by eliminating addiction potential while providing targeted therapeutic relief

In still another embodiment, the apparatus of the invention may be used to treat fabry disease with ovary cells. Fabry Disease is a rare inherited (X-linked) genetic lysosomal storage disease, which can result in a wide range of signs and symptoms that affect many parts of the body. Patients can experience episodes of full body or localized pain to the extremities, or life-threatening kidney complications or cardiac complications can occur when glycolipids accumulate. Other symptoms that affect quality of life include dermatological, gastrointestinal, auricular, and ocular manifestations. Fabry disease is a result of a deficiency of the enzyme alpha galactosidase (a-GAL A encoded by GLA). Current treatment involves an enzyme replacement therapy (ERT) designed to provide the enzyme the patient is missing as a result of the genetic malfunction. This drug called Fabrazyme has an annual cost of about $200,000 per patient in 2012. Lysosomal replacement enzymes are essential therapeutic options for rare congenital lysosomal enzyme deficiencies, but enzymes in clinical use are only partially effective due to short circulatory half-life and inefficient biodistribution. It has been discovered that cell lines are capable of producing lysosomal enzymes with N-glycans custom designed to affect key glycan features guiding cellular uptake and circulation. The apparatus of the invention can deliver engineered cells capable of production and secretion of lysosomal enzymes, in a manner that maintains cell viability and function. The apparatus of the invention can be used as a cell implantation therapy that protects transplanted protein-producing cells from the host immune system. Implanted cells result in fewer treatments than the standard of care (multiple injections) which improves patient compliance as well as reduces plasma, urinary sediment and tissue levels of Gb3, decreases the frequency of pain, improves or stabilizes cardiac and renal function, and increases the success rate of enzyme-replacement therapy in treating Fabry disease.

In yet another embodiment, the apparatus of the invention may be used to treat hearing loss with neurothrophins (BDNF & NT-3). Hearing loss affects approximately 500 million people worldwide mostly related to the death of either inner ear hair cells (HCs) or spiral ganglion neurons (SGNs) related to aging, injury, ototoxic drugs, acoustic trauma, or disease. Mechanistic studies have demonstrated the regenerative potential of HCs and SGNs and uncovered growth factors capable of either preventing SGN and hair-cell death or stimulating hair-cell regeneration. The apparatus of the invention can deliver engineered cells capable of production and secretion of neurotrophic factor (or similar), via an inner ear delivery system in a manner that maintains cell viability and function. The apparatus of the invention can be used as a cell implantation therapy that protects transplanted neurotrophic factor-producing cells from the host immune system. Cell-based drug delivery provides an alternative approach to chronically treat inner ear neurons at physiologic neuroprotective concentrations and has the comparative advantage of non-invasive reloading which is likely necessary for life-long delivery of these factors. The apparatus of the invention alone or in combination with cochlear implants enhances therapeutic value in prevention and treatment of hearing impairment.

In still another embodiment, the apparatus of the invention may be used to treat hemophilia with human factor IX secreting cells. Hemophilia A, which is also called factor VIII (FVIII) deficiency, is a genetic disorder caused by deficient or defective factor VIII, a dotting protein. It is an X-linked recessive disorder that can be inherited or arise from spontaneous mutation (about ⅓ of cases). According to the US Centers for Disease Control and Prevention, hemophilia occurs in approximately 1 in 5,000 live births and there are about 20,000 people with hemophilia in the US. People with hemophilia often bleed longer than other people. Bleeds can occur internally, into joints and muscles, or externally, from minor cuts, dental procedures, or trauma. How frequently a person bleeds and the severity of those bleeds depends on how much FVIII is in the plasma. Normal plasma levels of FVIII range from 50% to 150%. Levels below 50%, or half of what is needed to clot, determine a person's systems. The main medication to treat hemophilia is concentrated FVIII product, called clotting factor or simply factor. Recombinant factor products, which are developed in the lab through the use of DNA technology, preclude the use of human-derived pools of donor sourced plasma. Approximately 75% of the hemophilia community takes a recombinant FVIII product which are infused intravenously through a vein in the arm or port in the chest. A number of cell types have been demonstrated functional FVIII production, for example liver sinusoidal endothelial cells (LSECs). The apparatus of the invention can deliver isolated LSECs, or engineered cells capable of production and secretion of FVIII, in a manner that maintains cell viability and function. The apparatus of the invention may be used as a cell implantation therapy that protects transplanted FVIII-producing cells from the host immune system. Implanted cells result in fewer treatments than the standard of care (multiple injections) which improves patient compliance as well as maintains enough clotting factor to prevent serious bleeds.

The apparatus of the invention has been shown to provide foreign bodies with a suitable environment to flourish and macro-encapsulating FVIII plasma into the apparatus of the invention provides a suitable alternative.

In another embodiment, the apparatus of the invention may be used to treat renal failure with bacteria for the elimination of urea. Renal failure or chronic kidney disease (CKD) occurs in which there is a gradual loss of kidney function over a period of months or years. Causes of chronic kidney disease include diabetes, high blood pressure, glomerulonephritis, and polycystic kidney disease and risk factors include a family history to the condition. CKD affected 753,000,000 people globally in 2016. The apparatus of the invention enables a therapeutic approach using human renal progenitor cells (hRPCs) or renal mesenchymal stromal cells (MSCs) to trigger the kidney to repair itself. The apparatus of the invention can deliver isolated RPCs, or engineered cells capable of production and secretion of RPC- MSC-factors, in a manner that maintains cell viability and function. The apparatus of the invention can be used as a cell implantation therapy that protects transplanted RPCs from the host immune system and allows for targeted local delivery of these factors (e.g. intraparenchymal). Implanted cells protect the kidney from further degeneration and support renal healing which dramatically decreases the need for dialysis or renal transplant by secreting growth factors that contribute to tissue repair and kidney regeneration.

In yet another embodiment, the apparatus of the invention may be used to treat liver failure or chronic liver disease with the implantation of hepatocytes. Chronic liver disease (CLD) encompasses a large number of conditions having different etiologies and existing on a continuum between hepatitis infection and cirrhosis. Chronic liver disease is the 10th leading cause of mortality in the US and is responsible for the deaths of more than 25,000 Americans each year. Liver inflammation is characterized by the destruction of a number of liver cells and the presence of inflammatory cells in the liver tissue and it can be caused by diseases that primarily attack the liver cells. Hepatic fibrosis is a reversible scarring response to chronic liver injury which produces inflammation initially.

Progression of liver inflammation leads to hepatic fibrosis followed by cirrhosis, liver cancer and liver failure. The apparatus of the invention enables a therapeutic approach using hepatocytes, hepatocyte-like cells, or mesenchymal stromal cells (MSCs) to trigger the liver to repair itself. The apparatus of the invention can deliver isolated hepatocytes, or engineered cells capable of production and secretion of hepatocyte-MSC-factors, in a manner that maintains cell viability and function. The apparatus of the invention is a cell implantation therapy that protects transplanted cells from the host immune system and allows for targeted local delivery of these factors (e.g. intraparenchymal). Implanted cells protect the liver from further degeneration and support restoration of liver function which dramatically decreases the need for transplant by secreting anti-inflammatory, anti-apoptotic, immunomodulatory, and pro-proliferative factors that contribute to tissue repair and liver regeneration.

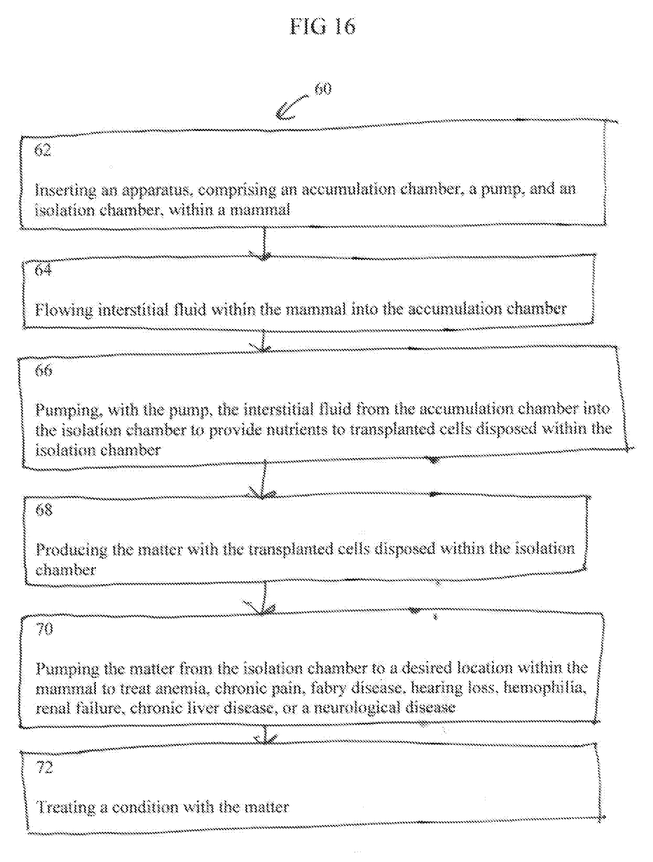

FIG. 16 illustrates one embodiment of a method 60 of producing and delivering matter within a mammal. The method 60 may utilize any embodiments of the apparatus of the invention disclosed herein. In other embodiments, the method 60 may utilize varying apparatus. Step 62 comprises inserting an apparatus, comprising an accumulation chamber, a pump, and an isolation chamber, within a mammal. In one embodiment, transplanted cells may be disposed in the isolation chamber prior to the apparatus being inserted into the mammal. In another embodiment, the transplanted cells may be inserted into the isolation chamber after inserting the apparatus into the mammal with a needle or other mechanisms. The transplanted cells may be inserted into the isolation chamber through a septum or port of the apparatus. In other embodiments, the transplanted cells may be inserted into the isolation chamber using different access members. In one embodiment, step 62 may comprise inserting the apparatus into a subcutaneous tissue of the mammal.

Step 64 comprises flowing interstitial fluid within the mammal into the accumulation chamber. In one embodiment, step 64 may comprise a tissue prevention member preventing tissue from blocking the accumulation. In one embodiment, the tissue prevention member may comprise a porous, liquid permeable interstitial fluid filter, screen, or mesh having a pore size in a range of 1 to 100 microns. In another embodiment, the tissue prevention member may comprise a tortuous path of spaced-apart posts. In still other embodiments, the tissue prevention member may vary. In another embodiment, step 64 may comprise a flow of the interstitial fluid being controlled with at least one one-way check valve.

Step 66 comprises pumping, with the pump, the interstitial fluid from the accumulation chamber into the isolation chamber to provide nutrients to transplanted cells disposed within the isolation chamber. In one embodiment, step 66 may comprise a flow of the interstitial fluid being controlled with at least one one-way check valve. Step 68 comprises producing the matter with the transplanted cells disposed within the isolation chamber. Step 70 comprises pumping the matter from the isolation chamber to a desired location within the mammal to treat anemia, chronic pain, fabry disease, hearing loss, hemophilia, renal failure, chronic liver disease, or a neurological disease. In one embodiment, step 70 may comprise a flow of the matter being controlled with at least one one-way check valve. In another embodiment, step 70 may comprise pumping the matter from the isolation chamber, through a catheter of the apparatus, to the desired location. The desired location may comprise a peritoneal cavity or a portal vein of the mammal. In another embodiment, the desired location may vary.

Step 72 may comprise treating a condition with the matter. In one embodiment, step 72 may comprise treating the anemia with the matter, the transplanted cells may comprise isolated renal peritubular Erythropoietin-producing cells or engineered Erythropoietin-producing cells, and the matter produced by the transplanted cells may comprise Erythropoietin. In another embodiment, step 72 may comprise treating the chronic pain with the matter, the transplanted cells may comprise chromaffin cells which produce the matter comprising amines and peptides, or the transplanted cells may comprise engineered cells which produce the matter comprising opioid peptides and catecholamines. In another embodiment, step 72 may comprise treating the fabry disease with the matter, the transplanted cells may comprise engineered lysosomal-enzyme producing cells, and the matter produced by the transplanted cells may comprise lysosomal-enzyme.

In another embodiment, step 72 may comprise treating the hearing loss with the matter, the transplanted cells may comprise engineered neurotrophic-factor producing cells, and the matter produced by the transplanted cells may comprise neurotrophic-factor. In another embodiment, step 72 may comprise, treating the hemophilia with the matter, the transplanted cells may comprise isolated liver sinusoidal endothelial cells or engineered factor VIII producing cells, and the matter produced by the transplanted cells may comprise factor VIII. In another embodiment, step 72 may comprise treating the renal failure with the matter, the transplanted cells may comprise isolated renal progenitor cells, engineered renal progenitor producing cells, or engineered mesenchymal stromal producing cells, and the matter produced by the transplanted cells may comprise renal progenitor cell factors or mesenchymal stromal cell factors. In another embodiment, step 72 may comprise treating the chronic liver disease with the matter, the transplanted cells may comprise isolated hepatocytes, engineered hepatocyte producing cells, or engineered mesenchymal stromal producing cells, and the matter produced by the transplanted cells may comprise hepatocytes, hepatocyte factors, or mesenchymal stromal factors.

In another embodiment, step 72 may comprise treating the neurological disease with the matter, the transplanted cells may comprise isolated neural stem cells, engineered neural stem producing cells, or engineered mesenchymal stromal producing cells, and the matter produced by the transplanted cells may comprise neural stem cells, neural stem cell factors, or mesenchymal stromal factors. In other embodiments, step 72 may further vary to use varying transplanted cells to produce varying matter to treat varying types of conditions.

In still other embodiments, one or more steps of the method 60 may be modified in substance or order, one or more of the steps may not be followed, or one or more additional steps may be added in any order.

1. A method of producing and delivering matter within a mammal comprising:
inserting an apparatus, comprising an accumulation chamber, a pump, and an isolation chamber, within a mammal;
flowing interstitial fluid within the mammal into the accumulation chamber;
pumping, with the pump, the interstitial fluid from the accumulation chamber into the isolation chamber to provide nutrients to transplanted cells disposed within the isolation chamber;
producing the matter with the transplanted cells disposed within the isolation chamber; and
pumping the matter from the isolation chamber to a desired location within the mammal to treat anemia, chronic pain, fabry disease, hearing loss, hemophilia, renal failure, chronic liver disease, or a neurological disease.

2. The method of claim 1 wherein the inserting the apparatus within the mammal comprises inserting the apparatus into a subcutaneous tissue of the mammal.

3. The method of claim 1 further comprising a tissue prevention member preventing tissue from blocking the accumulation chamber.

4. The method of claim 3 wherein the tissue prevention member comprises a porous, liquid permeable interstitial fluid filter, screen, or mesh.

5. The method of claim 4 wherein the tissue prevention member has a pore size in a range of 1 micron to 100 microns.

6. The method of claim 3 wherein the tissue prevention member comprises a tortuous path.

7. The method of claim 6 wherein the tortuous path comprises a plurality of spaced-apart posts.

8. The method of claim 1 further comprising controlling a flow of the interstitial fluid within the apparatus using at least one one-way check valve.

9. The method of claim 1 further comprising inserting the transplanted cells into the isolation chamber prior to the apparatus being inserted the mammal.

10. The method of claim 1 further comprising inserting the transplanted cells into the isolation chamber after inserting the apparatus into the mammal.

11. The method of claim 10 further comprising inserting the transplanted cells into the isolation chamber using a needle.

12. The method of claim 1 further comprising inserting the transplanted cells into the isolation chamber through a septum or port.

13. The method of claim 1 wherein the pumping the matter from the isolation chamber to the desired location comprises pumping the matter from the isolation chamber, through a catheter of the apparatus, to the desired location.

14. The method of claim 13 wherein the desired location is a peritoneal cavity or a portal vein of the mammal.

15. The method of claim 1 further comprising treating the anemia with the matter.

16. The method of claim 15 wherein the transplanted cells comprise isolated renal peritubular Erythropoietin-producing cells or engineered Erythropoietin-producing cells, and the matter produced by the transplanted cells comprises Erythropoietin.

17. The method of claim 1 further comprising treating the chronic pain with the matter.

18. The method of claim 17 wherein the transplanted cells comprise chromaffin cells which produce the matter comprising amines and peptides, or the transplanted cells comprise engineered cells which produce the matter comprising opioid peptides and catecholamines.

19. The method of claim 1 further comprising treating the fabry disease with the matter.

20. The method of claim 19 wherein the transplanted cells comprise engineered lysosomal-enzyme producing cells, and the matter produced by the transplanted cells comprises lysosomal-enzyme.

21. The method of claim 1 further comprising treating the hearing loss with the matter.

22. The method of claim 21 wherein the transplanted cells comprise engineered neurotrophic-factor producing cells, and the matter produced by the transplanted cells comprises neurotrophic-factor.

23. The method of claim 1 further comprising treating the hemophilia with the matter.

24. The method of claim 23 wherein the transplanted cells comprise isolated liver sinusoidal endothelial cells or engineered factor VIII producing cells, and the matter produced by the transplanted cells comprises factor VIII.

25. The method of claim 1 further comprising treating the renal failure with the matter.

26. The method of claim 25 wherein the transplanted cells comprise isolated renal progenitor cells, engineered renal progenitor producing cells, or engineered mesenchymal stromal producing cells, and the matter produced by the transplanted cells comprises renal progenitor cell factors or mesenchymal stromal cell factors.

27. The method of claim 1 further comprising treating the chronic liver disease with the matter.

28. The method of claim 27 wherein the transplanted cells comprise isolated hepatocytes, engineered hepatocyte producing cells, or engineered mesenchymal stromal producing cells, and the matter produced by the transplanted cells comprises hepatocytes, hepatocyte factors, or mesenchymal stromal factors.

29. The method of claim 1 further comprising treating the neurological disease with the matter.

30. The method of claim 29 wherein the transplanted cells comprise isolated neural stem cells, engineered neural stem producing cells, or engineered mesenchymal stromal producing cells, and the matter produced by the transplanted cells comprises neural stem cells, neural stem cell factors, or mesenchymal stromal cell factors.
